(19) United States
(12) Patent Application Publication
(10) Pub. No.: US 2004/0259851 A1
(43) Pub. Date: Dec. 23, 2004

(54) Methods of administering estrogens and progestins

(76) Inventor: Thomas W. Leonard, Wilmington, NC (US)

Correspondence Address: MYERS BIGEL SIBLEY & SAJOVEC, PO BOX 37428, RALEIGH, NC, 27627, US

(21) Appl. No.: 10/821,278

(22) Filed: Apr. 8, 2004

Related U.S. Application Data
(60) Provisional application No. 60/462,285, filed on Apr. 11, 2003.

(51) Int. Cl. A61K 31/56

(52) U.S. Cl. 514/170

(57) ABSTRACT

The present invention includes methods for treating endometrial hyperplasia and vasomotor symptoms through the administration of estrogenic compounds and progestational agents. The methods presented may include starting estrogen therapy with a progestational agent at a high dose, and then lowering the dose once therapy has been shown to be effective.

### CROSS REFERENCE TO RELATED APPLICATIONS

The present application claims priority to U.S. Provisional Application No. 60/462,285, filed Apr. 11, 2003, the disclosure of which is incorporated herein by reference in its entirety.

### FIELD OF THE INVENTION

The present invention generally relates to a method of treating hormonal deficiencies in women, particularly menopausal and post-menopausal women.

### BACKGROUND OF THE INVENTION

Menopause typically occurs in women during middle age and is often described as an ovarian shutdown. Menopause is usually associated with a profound decrease in circulating levels of estrogens. Currently, there are a large variety of disorders and conditions that are attributed to the reduction of estrogen levels. These disorders and conditions include hot flashes, dryness and atrophy of the vagina, parathesia, dyspareunia, osteoporosis, and an increase in cardiovascular disease. In an effort to reduce these disorders and conditions, estrogens are administered to women in a so-called “estrogen replacement therapy”. Estrogen replacement therapy continues to be the primary treatment of such disorders and conditions associated with menopause. Additionally, estrogens may also be used in postmenopausal women in the treatment of osteoporosis and to delay onset of or prevent cardiovascular disease and Alzheimer's disease.

One of the risks associated with the administration of estrogen replacement therapy is that women with intact uteri may develop endometrial hyperplasia. The term “endometrial hyperplasia” refers to the over stimulation of the lining of the uterus, which is a precursor to endometrial or uterine cancer. The development of endometrial hyperplasia is a significant issue with estrogen replacement therapy. For example, it has been observed in U.S. Pat. No. RE 36,247 to Plunkett, et al., and U.S. Pat. No. 5,043,331 to Hirvonen, that the co-administration of progestin can blunt the effect of estrogens. However, side effects often still occur with this co-administration. Thus, it may be desirable to have an estrogen replacement therapy in which the potential side effects relating to such therapy were reduced.

As noted above, vasomotor hot flashes are a common symptom in women during menopause. Vasomotor hot flashes have also been associated with men who have undergone androgen-deprivation therapy for prostate cancer. Various studies have suggested that megestrol acetate, a progestational agent, can decrease the frequency of hot flashes. Loprinzi et al. “Megestrol Acetate for the Prevention of Hot Flashes”, The New England Journal of Medicine, 331:347-352 (1994). A nonblinded study reported that megestrol acetate in daily doses of 20, 40, and 80 mg decreased menopausal hot flashes by 80, 89, and 98 percent, respectively. Erlik et al. “Effect of megestrol acetate on flushing and bone metabolism in post-menopausal women”, Maturitas, 3:167-172 (1981). However, a progestin administered in large doses, together with large amounts of a synthetic estrogen, induces changes in blood lipids which may promote arteriosclerotic changes and have been implicated in the appearance of strokes and myocardial infarction among women taking oral contraceptives in their later reproductive years. Plunkett, Am. J. Obs/Gyn. 142, 6, 747-751 (1982). Thus it may be desirable to relieve vasomotor symptoms through alternative methods of therapy.

### SUMMARY OF THE INVENTION

The present invention discloses methods of treating vasomotor symptoms, endometrial hyperplasia, and menopause. The present invention also discloses various methods of estrogen therapy and hormonal replacement therapy. The methods employed by the present invention include administering a low dose of a progestin with estrogen therapy.

The present invention includes methods of treating endometrial hyperplasia comprising administering a therapeutic amount of an estrogenic compound to a subject; and administering a therapeutic amount of a progestational agent of less than 20 mg.

The present invention also discloses methods of treating endometrial hyperplasia comprising administering a therapeutic amount of an estrogenic compound to a subject; and administering a therapeutic amount of a progestational agent of less than 20 mg, wherein said progestational agent is megestrol acetate.

Additionally, the present invention recites methods of treating vasomotor symptoms comprising administering a first dose of a therapeutic amount of an estrogenic compound to a subject; administering a second dose of a therapeutic amount of an estrogenic compound at a later time period to the subject, said second dose comprising a lower dosage of said therapeutic amount of an estrogenic compound than said first dose; and administering a therapeutic amount of a progestational agent of less than 20 mg.

The present invention also recites methods of treating menopause comprising administering a therapeutic amount of an estrogenic compound to a subject; and administering a therapeutic amount of a progestational agent of less than 20 mg.

Additionally, the present invention may include methods of treating endometrial hyperplasia comprising administering a dose of a therapeutic amount of an estrogenic compound to a subject; administering a dose of a therapeutic amount of less than 20 mg of megestrol acetate to a subject; and administering a second dose of a therapeutic amount of megestrol acetate at a later time period to the subject, said second dose comprising a lower dosage of said therapeutic amount of megestrol acetate than said first dose.

The present invention also discloses methods of treating vasomotor symptoms comprising administering a dose of a therapeutic amount of an estrogenic compound to a subject; administering a dose of a therapeutic amount of less than 20 mg of megestrol acetate to a subject; and administering a second dose of a therapeutic amount of megestrol acetate at a later time period to the subject, said second dose comprising a lower dosage of said therapeutic amount of megestrol acetate than said first dose.

### DETAILED DESCRIPTION OF THE EMBODIMENTS

The invention will now be described with reference to the embodiments set forth herein. These embodiments are intended to illustrate the invention and are not meant to limit the scope of the invention.

In one aspect, the invention relates to a method of administering a pharmaceutical composition. The pharmaceutical composition comprises a therapeutically effective amount of an estrogenic compound, a therapeutically effective amount of a progestational agent and a pharmaceutically acceptable carrier. The composition may also comprise an androgenic compound, wherein the androgenic compound is preferably a non-aromatizing androgen.

A “therapeutically effective” amount as used herein is an amount of an estrogenic compound that is sufficient to treat hormonal deficiencies in a subject. The therapeutically effective amount will vary with the age and physical condition of the patient, the severity of the treatment, the duration of the treatment, the nature of any concurrent treatment, the pharmaceutically acceptable carrier used and like factors within the knowledge and expertise of those skilled in the art. Pharmaceutically acceptable carriers are preferably solid dosage forms such as tablets or capsules. Liquid preparations for oral administration may be also be used and may be prepared in the form of syrups or suspensions, e.g., solutions containing an active ingredient, sugar, and a mixture of ethanol, water, glycerol, and propylene glycol. If desired, such liquid preparations may contain coloring agents, flavoring agents, and saccharin. Thickening agents such as carboxymethylcellulose may also be used. Additionally, transdermal patches and other acceptable carriers, the selection of which are known in the art.

Estrogen levels are related to the general physiological health of postmenopausal women. Estrogens exert positive central nervous system (CNS) effects on hot flashes, and improve nerve transmission which is believed to delay various types of dementia. They have positive cardiovascular effects by improving lipid levels and promoting vasodilation and relaxation. They also contribute to health of the vagina, provide local vasodilation effects and stimulate mucous production. Suitable estrogenic compounds include estrone, 17α-estradiol, 17β-estradiol, equilin, 17α-dihydroequilin, 17β-dihydroequilin, equilenin, 17α-dihydroequilenin, 17β-dihydroequilenin, Δ^{8,9}-dehydroestrone, 17α Δ^{8,9}-dehydroestradiol, 17β Δ^{8,9}-dehydroestradiol, 6-OH equilenin, 6-OH 17-α dihydroequilenin, ethinyl estradiol, estradiol valerate, 6-OH 17-β dihydroequilenin, and mixtures, conjugates and salts thereof, and the estrogen ketones and their corresponding 17-α and 17-β hydroxy derivatives. The estrogenic compounds may also be present as conjugated estrogens. A composition of these compounds may include a mixture that comprises salts of conjugated estrone, conjugated equilin, conjugated Δ^{8,9}-dehydroestrone, conjugated 17α-estradiol, conjugated 17α-dihydroequilin, conjugated 17β-dihydroequilin, conjugated 17β-estradiol, conjugated equilenin, conjugated 17α-dihydroequilenin, and conjugated 17β-dihydroequilenin. Another composition may include estrone, equilin, 17α-dihydroequilin, 17β-dihydroequilin and 17α-estradiol.

Approximately 1.0 mg of 17-β estradiol is equivalent to 0.625 mg of conjugated estrogens. The conjugates may be various conjugates understood by those skilled in the art, including, but not limited to, glucuronide, sulfate, phosphate and pyrophosphate. The most preferred estrogen conjugates are estrogen sulfates. The estrogenic compounds may also be present as salts of estrogens conjugates. The salts may be various salts understood by those skilled in the art, including, but not limited to, sodium salts, calcium salts, magnesium salts, lithium salts, and piperazine salt. The most preferred salts are sodium salts. The estrogenic compounds can be derived from natural and synthetic sources. Preferably, the therapeutically effective amount of estrogenic compound is about 0.05 to about 5 mg, and preferably about 0.25 to about 3 mg based on oral dose equivalents of estradiol. Even more preferable the therapeutically effective amount of the estrogenic compound is about 0.45 to about 2 mg. Even more preferable the therapeutically effective amount of the estrogenic compound is about 0.625 to about 1.5 mg.

As previously stated, androgenic compounds may be combined with the estrogenic compounds. Suitable androgenic compounds include both aromatizing and non-aromatizing compounds. Non-aromatizing compounds include as oxandrolone, oxymetholone, stanozolol, stanozolone, danazol, pharmaceutically acceptable esters and salts thereof, and combinations of any of the foregoing. Aromatizing compounds included, but are not limited to, androsterone, androstenediol, 4-androstene-3, 17-dione, and (3,5)-androst-16-en-3-ol. Preferably, the therapeutically effective amount of the androgenic compound is about 0.25 to about 10 mg. For women suffering from androgen deficiency the oral dosage equivalents of oxandrolone is about 0.5 to 4 mg of an androgenic compound per day.

Additionally, as previously stated, a progestational agent may be used in combination with the estrogenic compound. Examples of progestational agents are set forth in U.S. Pat. No. Re. 36,247 to Plunkett et al., the contents of which are incorporated by reference in their entirety. Examples include, but are not limited to, laevo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynoestrenol, quingestanol acetate, medrogestone, norgestrienone, dimethisterone, ethisterone, and cyproterone acetate. Another progestational agent is megestrol acetate. Megestrol acetate has the chemical name Pregna-4,6-diene-3,20-dione, 17-(acetyloxy)-6-methyl-, a formula of C₂₄H₃₂O₄ and a molecular weight of 384.51.

Megestrol acetate may be administered in doses ranging from 0.1 to less than 20 mg. More preferably the therapeutically effective amount of megestrol acetate is from about 0.25 to 18 mg. Even more preferable the therapeutically effective amount of megestrol acetate is from about 1.0 to 16 mg. Even more preferable the therapeutically effective amount of megestrol acetate is from about 1.5 to 12 mg. Even more preferable the therapeutically effective amount of megestrol acetate is from about 3 to 8 mg.

The estrogen formulations of the present invention may be, for example, in the form of tablets; effervescent tablets; pills; powders; elixirs; suspensions; emulsions; solutions; syrups; soft and hard gelatin capsules; transdermal patches; topical gels, creams and the like; vaginal suppositories; sterile injectable solutions; and sterile packaged powders, sublingual tablets, buccal tablets and buccal adhesive systems.

In certain embodiments, the drug product is present in a solid pharmaceutical composition that may be suitable for oral administration. A solid composition of matter according to the present invention may be formed and may be mixed with and/or diluted by an excipient. The solid composition of matter may also be enclosed within a carrier which may be, for example, in the form of a capsule, sachet, tablet, paper, or other container. When the excipient serves as a diluent, it may be a solid, semi-solid, or liquid material which acts as a vehicle, carrier, or medium for the composition of matter.

Various suitable excipients will be understood by those skilled in the art and may be found in the National Formulary, 19: 2404-2406 (2000), the disclosure of pages 2404 to 2406 being incorporated by reference herein in their entirety. Examples of suitable excipients include, but are not limited to, starches, gum arabic, calcium silicate, microcrystalline cellulose, methacrylates, shellac, polyvinylpyrrolidone, cellulose, water, syrup, and methylcellulose. The drug product formulations can additionally include lubricating agents such as, for example, talc, magnesium stearate and mineral oil; wetting agents; emulsifying and suspending agents; preserving agents such as methyl- and propyl hydroxybenzoates; sweetening agents; or flavoring agents. Polyols, buffers, and inert fillers may also be used. Examples of polyols include, but are not limited to, mannitol, sorbitol, xylitol, sucrose, maltose, glucose, lactose, dextrose, and the like. Suitable buffers encompass, but are not limited to, phosphate, citrate, tartarate, succinate, and the like. Other inert fillers which may be used encompass those which are known in the art and are useful in the manufacture of various dosage forms. If desired, the solid formulations may include other components such as bulking agents and/or granulating agents, and the like. The drug products of the invention may be formulated so as to provide quick, sustained, or delayed release of the active ingredient after administration to the patient by employing procedures well known in the art.

To form tablets for oral administration, the composition of matter of the present invention may be made by a direct compression process. In this process, the active drug ingredients may be mixed with a solid, pulverant carrier such as, for example, lactose, saccharose, sorbitol, mannitol, starch, amylopectin, cellulose derivatives or gelatin, and mixtures thereof, as well as with an antifriction agent such as, for example, magnesium stearate, calcium stearate, and polyethylene glycol waxes. The mixture may then be pressed into tablets using a machine with the appropriate punches and dies to obtain the desired tablet size. The operating parameters of the machine may be selected by the skilled artisan. Alternatively, tablets for oral administration may be formed by a wet granulation process. Active drug ingredients may be mixed with excipients and/or diluents. The solid substances may be ground or sieved to a desired particle size. A binding agent may be added to the drug. The binding agent may be suspended and homogenized in a suitable solvent. The active ingredient and auxiliary agents may also be mixed with the binding agent solution. The resulting dry mixture is moistened with the solution uniformly. The moistening typically causes the particles to aggregate slightly, and the resulting mass is pressed through a stainless steel sieve having a desired size. The mixture is then dried in controlled drying units for the determined length of time necessary to achieve a desired particle size and consistency. The granules of the dried mixture are sieved to remove any powder. To this mixture, disintegrating, antifriction, and/or anti-adhesive agents are added. Finally, the mixture is pressed into tablets using a machine with the appropriate punches and dies to obtain the desired tablet size. The operating parameters of the machine may be selected by the skilled artisan.

If coated tablets are desired, the above prepared core may be coated with a concentrated solution of sugar or cellulosic polymers, which may contain gum arabic, gelatin, talc, titanium dioxide, or with a lacquer dissolved in a volatile organic solvent or a mixture of solvents. To this coating various dyes may be added in order to distinguish among tablets with different active compounds or with different amounts of the active compound present. In a particular embodiment, the active ingredient may be present in a core surrounded by one or more layers including enteric coating layers.

Soft gelatin capsules may be prepared in which capsules contain a mixture of the active ingredient and vegetable oil. Hard gelatin capsules may contain granules of the active ingredient in combination with a solid, pulverulent carrier, such as, for example, lactose, saccharose, sorbitol, mannitol, potato starch, corn starch, amylopectin, cellulose derivatives, and/or gelatin.

In one embodiment of the invention, the formulation may be in the form of orally-administered tablets which contain the composition of matter of the present invention as set forth herein along with the following inactive ingredients: calcium phosphate tribasic, calcium sulfate, carnauba wax, cellulose, glyceryl monooleate, lactose, magnesium stearate, methylcellulose, pharmaceutical glaze, polyethylene glycol, stearic acid, sucrose, and titanium dioxide. Such ingredients may be present in amounts similar to those present in Premarin® (conjugated estrogens tablets, USP) made commercially available by Wyeth-Ayerst Laboratories of Philadelphia, Pa. Tablets employing the active ingredients of the invention may contain excipients similar to those contained in the 0.3 mg, 0.625 mg, and 1.25 mg tablets of Premarin® (conjugated estrogens tablets, USP).

Other methods may utilize an aqueous medium which contains an active ingredient or ingredients, a quantity of one or more surfactants sufficient to dissolve or suspend said active ingredients uniformly throughout the medium and other manufacturing additives as known to the art. The latter include granulating-binding agents such as gelatin; natural gums, such as acacia, tragacanth; starches, sodium alginate, sugars, polyvinylpyrrolidone; cellulose derivatives such as hydroxypropylmethylcellulose, polyvinyloxoazolidones; pharmaceutical fillers such as lactose, microcrystalline cellulose, dicalcium phosphate, tricalcium phosphate, calcium sulfate, dextrose, mannitol, sucrose; tabletting lubricants if needed such as calcium and magnesium stearate, stearic acid, talc, sterotex (alkaline stearate).

The components may then be granulated, the resulting granules are dried, sieved and compressed into tablets or filled into capsules. Other oral product forms may be similarly prepared by art methods such as chewable tablets, lozenges, troches, sustained or delayed release products or suspensions.

The active ingredients comprise any medicament which has a low effective dose such as those below 20 mg per dosage unit. Most useful are those medicaments having a steroidal nucleus, the cyclopentanoperhydrophenanthrene ring system, in their chemical structures such as the estrogens or progestins.

Examples of the former are ethinylestradiol, estrone, mestranol, 17-alpha-ethinyl estradiol-3-methylether esterified estrogens, and, especially estradiol, methyl testosterone. The dosage amounts and indications of these and other active ingredients are those described in the literature such as the Physician's Desk Reference.

The progestins are megestrol actetate 3-ketodesogestrel, desogestrel, levo-desogestrel, norgestrel, gestodene, mestranol, norethindrone, norethindrone acetate.

Other medications known to the art which are used in low doses are spironolactone, digoxin, glipizide, estazolam, clorazepate dipotassium, albuterol sulfate, clonidine HCL, alprazolam.

The term “aqueous medium” for the second ingredient of one of the embodiments of the invention is used within the custom of the pharmaceutical art. Primarily, it connotes a water medium, with added water-miscible solvents such as isopropanol or ethanol when needed, to support the active ingredient or pharmaceutical aids.

A third potential ingredient of the present invention may include a surfactant acceptable in pharmaceutical manufacturing practice and selected from the three categories of surfactants: cationic, anionic and non-ionic compounds. Exemplary of useful surfactants are sodium lauryl sulfate, sorbitan monolaurate, sorbitan monostearate, polysorbate 80, polysorbate 60, poloxamer 407, poloxamer 188 (polyoxethylene, polyoxypropylene block polymers), polyoxyl 20 cetostearyl ether, dioctyl sodium sulfosuccinate, dioctyl calcium sulfosuccinate, nonoxynol, benzalkonium chloride, sorbitan monooleate.

The quantity of surfactant in the granulating mixture is enough to be non-toxic and to support the steroidal active ingredient in solution or suspension. Usually, this means very small, almost catalytic, quantities, such as less than 0.01% by weight. Applicant has devised a simple test procedure for determining the applicability of a selected surfactant for this process. Details are presented below.

Other pharmaceutically acceptable additives are used in the first step granulation but are not considered critical to this invention. These include binding-granulating agents such as polyacrylamides, polyvinyloxoazolidones, sucrose, and sodium carboxymethylcellulose; fillers such as lactose, talc, cellulosics, dibasic calcium phosphate, starches; disintegrants if a tablet or capsule is formed, such as croscarmellose sodium, starch, sodium carboxymethyl starch, veegum, ion exchange resins (amberlite), sodium bicarbonate; or lubricants for tablet compression such as polyethylene glycol 4000 and 5000, hydrogenated vegetable oils, light mineral oil.

The practice of this invention depends on the novelty and practical benefits of using a low dose medicament, a pharmaceutically acceptable quantity of surfactant and an aqueous medium. Ingredients include, but are not limited to estradiol, sodium lauryl sulfate and water from a povidone solution. The therapeutic utility is demonstrated by oral administration of such a dosage unit from 1-5 times daily to a subject in need of treatment, for example for menopausal abnormalities.

In practice, the estradiol is suspended in a 1% povidone solution containing a trace (0.005%) of surfactant. The aqueous suspension is blended with fillers and granulated in a granulating vessel. The granulation is dried, screened and blended with fillers, disintegrants and lubricants. The granulation is then compressed into tablets.

Alternatively, the dried granules may be filled into a capsule. Where extended or delayed release of the low dose medicament is desired the granules or capsule may be coated as known to the art.

One procedure that may be followed to produce the tablets of the present invention may include the following. 1. Suspend the estradiol in a 1% povidone solution in which 0.005% sodium lauryl sulfate has been dispersed. 2. Blend the Cal-Star and lactose until homogeneous. 3. Granulate the blend from Step 2 with the suspension of estradiol in povidone solution from Step 1. 4. Dry the above granulation. 5. Screen and blend the dried granulation from Step 4 with the other ingredients. 6. Compress the blend from Step 5 into 164 mg tablets. Each tablet containing 2 mg of estradiol.

Another protocol for Screening Surfactant for Low Dose Drug Suspensions follows. I. Prepare a 1% povidone stock solution in water. II. Prepare reference solution of 1% povidone—0.005% sodium lauryl sulfate (SLS). A. Prepare a 14.3% w/w solution with the SLS solution and estradiol. B. Prepare a 14.3% w/w solution with the SLS solution and spironolactone. C. Compare the estradiol solution and the spironolactone solution. If they have the same appearance, spironolactone can be used as the model drug and estradiol can be used for a check. III. Use the stock povidone solution to prepare solutions with the other surfactants to be investigated, such as, but not limited to, dioctyl sodium sulfosuccinate, dioctyl calcium sulfosuccinate, nonoxynol, benzalkonium chloride, sorbitan monooleate. A. Prepare 1% povidone—1% surfactant solutions. 1. Prepare 14.3% w/w solution with the surfactant and the steroidally derived drug. 2. Compare to reference solution. 3. If the surfactant/steroidal solution conforms to the reference solution: a. Dilute surfactant solution with 1% povidone solution in 0.5% increments; b. Determine the lowest concentration of surfactant that a flocculated suspension can be formed.

Liquid preparations for oral administration may be prepared in the form of syrups or suspensions, e.g., solutions containing an active ingredient, sugar, and a mixture of ethanol, water, glycerol, and propylene glycol. If desired, such liquid preparations may contain coloring agents, flavoring agents, and saccharin. Thickening agents such as carboxymethylcellulose may also be used.

In the event that the above formulations are to be used for parenteral administration, such a formulation may comprise sterile aqueous injection solutions, non-aqueous injection solutions, or both comprising the composition of matter of the present invention. When aqueous injection solutions are prepared, the composition of matter may be present as a water soluble pharmaceutically acceptable salt. Parenteral preparations may contain anti-oxidants, buffers, bacteriostats, and solutes which render the formulation isotonic with the blood of the intended recipient. Aqueous and non-aqueous sterile suspensions may include suspending agents and thickening agents. The formulations may be presented in unit-dose or multi-dose containers, for example sealed ampules and vials. Extemporaneous injection solutions and suspensions may be prepared from sterile powders, granules and tablets of the kind previously described.

In one embodiment of the present invention, the drug product is in the form of an injectable solution containing a predetermined amount (e.g., 25 mg) of the composition of matter in a sterile lyophilized cake which also contains lactose, sodium citrate, and simethicone. The pH of a solution containing the above ingredients may be adjusted using a suitable buffer (e.g., sodium hydroxide or hydrochloric acid). Reconstitution may be carried out according to known methods, e.g., using a sterile diluent (5 mL) containing 2 percent by volume benzyl alcohol in sterile water. A preferred injectable solution is similar to Premarin® Intravenous made commercially available by Wyeth-Ayerst Laboratories.

The composition of matter also may be formulated such that it may be suitable for topical administration (e.g., vaginal cream). These formulations may contain various excipients known to those skilled in the art. Suitable excipients may include, but are not limited to, cetyl esters wax, cetyl alcohol, white wax, glyceryl monostearate, propylene glycol, monostearate, methyl stearate, benzyl alcohol, sodium lauryl sulfate, glycerin, mineral oil, water, carbomer, ethyl alcohol, acrylate adhesives, polyisobutylene adhesives, and silicone adhesives.

The drug product may be in the form of a vaginal cream containing the composition of matter as set forth herein present in a nonliquefying base. The nonliquefying base may contain various inactive ingredients such as, for example, cetyl esters wax, cetyl alcohol, white wax, glyceryl monostearate, propylene glycol, monostearate, methyl stearate, benzyl alcohol, sodium lauryl sulfate, glycerin, and mineral oil. Such composition may be formulated similar to Premarin® Vaginal Cream made commercially available by Wyeth-Ayerst Laboratories.

Dosage units for vaginal or rectal administration may be prepared in the form of suppositories which may contain the composition of matter in a mixture with a neutral fat base polyethylene glycol, or they may be prepared in the form of gelatin-rectal capsules which contain the active substance in a mixture with a vegetable oil or paraffin oil.

In one embodiment of the present invention, an estrogenic compound comprising 0.05 to 3 mg of estrogens, preferably conjugated estrogens, and a progestational agent comprising 1.5 to 16 mg of a progestin may be administered to a subject. The estrogenic compound may be used to treat vasomotor symptoms including, but not limited to hot flashes, also known as hot flushes, cold flashes/flushes, night and day sweats, dry vagina, dry hair and skin, insomnia, bladder problems and moodiness. The estrogenic compound may also be used to treat menopause or may be used in conjunction with or as an estrogen replacement therapy or hormonal replacement therapy.

The methods used in the present invention may also include reducing the amount of an estrogen given to a subject by starting out administering a high dose of an estrogenic compound to a subject and then gradually lowering the dose once therapy has been effectively established. One skilled in the art will be able to use a number of permutations in which the dosage of the estrogenic compound may be lowered.

As stated above, the methods used in estrogen therapy in the present invention may include starting estrogen therapy at a high dose, and then lowering the dose once therapy has been shown to be effective. Preferably, the estrogenic compound is administered in a therapeutic amount to a subject in a first dose is sufficient to alleviate vasomotor symptoms. The first dose may be administered daily, continuously and uninterruptedly for an effective time period until such time that therapy has been effectively established, preferably two to twelve weeks, more preferably four to eight weeks. The therapeutic amount of the estrogenic compound for the first dose is typically 0.05 to 3 mg of estrogens, more preferably 0.6 to 1.25 mg of estrogens. After the cycle for the therapeutic amount of an estrogenic compound of a first dose is completed, a second dose of a therapeutic amount of an estrogenic compound is administered to a subject. This second dose comprises a lower dosage of the therapeutic amount of the estrogenic compounds than the first dose. Preferably, the therapeutic amount of the estrogenic compound in the second dose is 0.05 to 2.5 mg, and more preferably 0.25 to 0.5 mg per dose. The second dose is administered continuously and uninterruptedly until a time when all vasomotor symptoms and other symptoms relating to menopause have been alleviated and will not return.

Additionally, once therapy has been effectively established it may be possible to continue the step-down therapy as disclosed above by decreasing the amount of estrogenic compound in a third or fourth dose. One skilled in the art will be able to choose additional regimens based upon this information.

The methods, preparations and pharmaceutical products of the present invention may also provide for at least two or more dosage strengths of progestational agents over the course of the treatment period such that the dosages, when administered as provided herein, may result in an acceptable bleeding pattern. The initial dosing of the progestin may be relatively high to assist in inducing or establishing a nonproliferative endometrium. Typically, this effect may be evidenced by an absence of substantive mitotic activity. The dosing may be used to enhance the formation of nonproliferative endometrium and results in a reduction of random bleeding during the remainder of the treatment period. This dosage strength typically is administered for about 7 to about 120 days. This time period may be less than 7 days depending on the dosage. Administration of a high dosage may allow for a shorter initial period. The dosage amount of progestin is then either gradually reduced in a series of steps or is reduced in one step to a maintenance amount that is less than the initiation amount. The maintenance dose preferably is at least about 25% less than the initial dose and, most preferably, the maintenance amount is about half of the initiation dosage. More preferably the second dose is at least a 50% reduction than the initial dose of progestins. Subsequent doses may be greater than 50%, i.e., administering 20 mg in the first dose and dropping the dose all the way to 0.1 mg. At this point in the treatment period, the dosage amount is such that the nonproliferative or atrophic endometrium is maintained or continued. This dosage amount of progestin inhibits or decreases the potential for breakthrough bleeding and spotting, typical problems in traditional therapies. This dosage strength is typically administered for about two to four weeks for short-term therapies or may be administered indefinitely for longer therapies. The treatment period ends upon cessation of administration of the estrogen and progestin therapy.

In one embodiment of the invention, the amount of progestin is provided in an initiation step of the treatment period in an amount exhibiting progestin activity equivalent to less than 20 mg of megestrol acetate and is provided in a maintenance step during the treatment period in an amount exhibiting progestin activity equivalent to below about 10.0 mg of megestrol acetate. Preferably, the amount of progestin activity is reduced by at least 25%; most preferably the amount of progestin is 50% of the amount in the maintenance step as the amount in the initiation step. In another aspect of the invention, the amount of progestin preferably is decreased in a series of steps to the maintenance step wherein the progestin activity is about half the amount administered in the initiation dosage.

In another embodiment of the invention, the progestin is provided in an initiation step of the treatment in an amount exhibiting progestin activity equivalent to an oral dose of about 1 mg to about 20 mg of megestrol acetate and is provided in a maintenance step during the treatment period in an amount exhibiting progestin activity equivalent to an oral dose of about 0.5 to about 10 mg of megestrol acetate; provided, however, that the amount of progestin activity is reduced in the maintenance step by at least 25%; most preferably the amount of progestin is 50% the amount in the maintenance step as the amount in the initiation step. A third step is most preferably reduced by an additional 50% from the second step.

In another embodiment of the present invention, the amount of progestin when the amount of estrogen is about 0.625 mg, may be either about 6 mg or about 12 mg in the initiation step and about 3 mg or about 6 mg, respectively, in the remaining or maintenance step of the treatment period. When the amount of estrogen is about 0.45 mg, the amount of progestin is preferably 5 mg or 10 mg, respectively, in the initiation step of the treatment period and approximately 2.5 mg or 5 mg in the remaining step or maintenance step of the treatment period. All amounts of progestin are in terms of biological equivalence to oral doses of megestrol acetate and all amounts of estrogen are in terms of biological equivalence to oral doses of conjugated estrogens. One skilled in the art will be able to compare the dose equivalency tables should they choose a progestin outside of megestrol acetate.

Thus, the methods used in the present invention may include reducing the amount of a progestin given to a subject by starting out administering a high dose of a progestin agent to a subject and then gradually lowering the dose once therapy has been effectively established. One skilled in the art will be able to use a number of permutations in that the dosage of the progestin agent may be lowered. Additionally, once therapy has been effectively established it may be possible to continue the step-down therapy as disclosed above by decreasing the amount of progestin agent in a third or fourth dose. One skilled in the art will be able to choose additional regimens based upon this information.

The first dose may be administered daily, continuously and uninterruptedly for an effective time period until such time that therapy has been effectively established, preferably one week to two months, more preferably two to six weeks.

The initiation dosage amount of progestin may be sufficient to enhance formation of or may help establish a nonproliferative or atrophic endometrium. The treatment may further substantially induce bleeding and then obviate or reduce random bleeding. The maintenance dosage amount is sufficient and effective for continuing or maintaining the nonproliferative endometrium established by the initiation dosage of progestin. The maintenance dosage amount further inhibits and decreases breakthrough bleeding and spotting observed in traditional therapies.

The methods may be used for a number of treatments such as, but not limited to, vasomotor symptoms; atrophic vaginitis; osteoporosis; hypoestrogenism due to hypogonadism, castration, or other primary ovarian failure, among others. The administration of estrogen and progestin according to the present invention may be continuous for a short-term, for example, to treat vasomotor symptoms, or may be continuous for a long-term, for example for osteoporosis. One example of long-term use would be from the onset of menopause until death.

The pharmaceutical product of the invention may be provided in a variety of forms, such that the sequential dosage units may be easily accessible by a subject. For example, the pharmaceutical product may be provided as a pharmaceutical package containing the sequential dosages in an arrangement suitable for daily administration of the appropriate dosages of estrogen and progestin. The number of dosages in each package may depend on the therapy and whether it is a long-term therapy for hormone deficiencies, or a short-term therapy. Typically, the pharmaceutical product may include a kit or package with daily dosages arranged for proper sequential administration. The sequence or arrangement of the dosage units will correspond to the stages of daily administration.

The present invention is primarily concerned with the treatment of human subjects, but the invention also may be carried out on animal subjects, particularly mammalian subjects such as mice, rats, dogs, cats, livestock and horses for veterinary purposes, and for drug screening and drug development purposes.

The present invention is explained in greater detail in the Examples which follow. These examples are intended as illustrative of the invention and are not to be taken are limiting thereof.

EXAMPLES

### Example 1

A twelve-week study in postmenopausal women comparing proliferative changes in the endometrial lining following administration of conjugated estrogens in combination with 1.5. mg or 6 mg of a progetational agent was performed. Fourteen randomized patients were selected who were naturally or surgically postmenopausal, non-hysterectomized women between the ages of 21 and 65 years.

The patients received 0.625 mg of conjugated estrogens in combination with either 1.5 mg of a progestational agent or 6 mg of a progestational agent. Once daily for twelve weeks. Efficacy was assessed by endometrial biopsies to evaluate the proliferative changes in the endometrial lining. Biopsies were performed at screening, week 8 and week 12.

The endometrial biopsy results from this study indicated that the 6 mg dose of a progestational agent was highly effective in inhibiting adverse tissue changes within the endometrial lining within the twelve-week program. The 6 mg dose was effective in inhibiting estrogen-induced tissue changes in the endometrial lining within the twelve-week period. The patients of the study exhibited a notable lack of endometrial hyperplasia as determined by a biopsy of the uterine lining.

### Example 2

A total of 90 plasma samples were analyzed for their content of Megestrol Acetate by GC/MS.

Materials and Methods

Reference Compounds

In this study megestrol acetate was utilized and medroxyprogesterone acetate was utilized as an internal standard.

Preparation of the Megestrol Acetate Solutions

Stock solutions of megestrol acetate were prepared in 50 mL volumetric flasks by weighing 5.0 (5.0) mg megestrol acetate and dissolving it in 50 mL methanol to obtain a concentration of 100 ng/μL of the analyte. These solutions were labeled “megestrol acetate stock solution (SSI) 100 ng/μL in methanol” and “megestrol acetate stock solution (SS2) 100 ng/μL in methanol”. The first stock solution was used for preparation of the calibration standards, the second stock solution for preparation of the quality control samples.

Working solutions of megestrol acetate were prepared by diluting the corresponding stock solution with methanol. The stock solutions were stored in a freezer at −20° C.±5° C. for use within 12 months.

Preparation of the Internal Standard Solutions

The internal standard stock solution was prepared in a 100 mL volumetric flask by weighing 9.97 mg internal standard and dissolving it in 100 mL methanol to obtain a concentration of 100 ng/μL of the internal standard. This solution was labelled “IS stock solution 100 ng/μL in methanol”.

The IS working solution was prepared by diluting the IS stock solution with methanol to obtain the concentration of 0.200 ng/μL. The internal standard stock solution 5° C.±3° C. and the working solution were stored in a refrigerator at 5° C.±3° C.

Calibration Standards and Quality Control Samples

Calibration standards were prepared freshly for each run by spiking 25 1.1 L of the corresponding working solution to 1 mL of plasma. The working solutions were prepared from the stock solution SS1 as described below.

Table 1 below illustrates the preparation of the working solutions and calibration standards.

TABLE 1

| | | Diluted with | Spiking sol. | Resulting in |
|---|---|---|---|---|
| Volume | | Methanol to | [ng/μL] | cal. standard¹⁾ |
| [mL] | Of solution | [mL] | (label) | [ng/mL] |
| 0.4 | SS1 | 50 | 0.8 (AK 1) | 20.0 |
| 0.2 | 551 | 50 | 0.4 (AK 2) | 10.0 |
| 0.1 | SS1 | 50 | 0.2 (AK 3) | 5.00 |
| 5.0 | AK-1 | 50 | 0.08 (AK 4) | 2.00 |
| 2.5 | AK-1 | 50 | 0.04 (AK 5) | 1.00 |
| 1.25 | AK-1 | 50 | 0.02 (AK 6) | 0.500 |
| 1.0 | AK-2 | 50 | 0.008 (AK 7) | 0.200 |
| 0.5 | AK-2 | 50 | 0.004 (AK 8) | 0.100 |

¹⁾by dilution of 25 μL spiking solution in 1 mL matrix

The quality control samples (QCs) were prepared in the same way as the calibration standards. The working solutions were prepared from stock solution SS2. The QC samples were prepared at the beginning of measurement. After preparation, the QC samples were stored at the same conditions as the study samples.

Table 2 shows the preparation of the QC samples.

TABLE 2

| | | | Spiking Sol. | Resulting in |
|---|---|---|---|---|
| | | Diluted with | [ng/μL] | OC sample¹⁾ |
| Volume [mL] | Of solution | methanol to | (label) | [ng/mL] |
| 0.5 | SS2 | 50 | 1.0 (AQ-1) | — |
| 30 | AQ-1 | 50 | 0.6 (AQ-2) | 15.0 |
| 3 | AQ-1 | 50 | 0.06 (AQ-3) | 1.50 |
| 0.5 | AQ-1 | 50 | 0.01 (AQ-4) | 0.250 |

¹⁾by dilution of 25 μL spiking solution in 1 mL matrix

Reagents

The solvents and reagents were of the same type and quality as used during method validation. All solvents and reagents including water were of p.a. quality or better. The reagents used are included toluene, diisopropylether, methanol and sodium hydroxide.

Sample Work-Up and Measurement

For each sample, 1 mL of human plasma was aliquoted and spiked with 25 μL of the internal standard working solution. In the following, the samples were alkalized and extracted with a toluene/diisopropylether mixture. The resulting extracts were stored in a refrigerator until measurement. Just prior to measurement, the sample extracts were evaporated to dryness and reconstituted in toluene. 2 μL of the resulting solution were injected into the GC/MS system. The gas chromatograph was run in the temperature-programmed mode to achieve improved separation. Positive ions chemical ionization (PICI) was used for mass selective detection. Selected mass/charge ratios were m/z 385.2 for megestrol acetate and m/z 387.2 for the internal standard.

Results

The results of all study samples are listed in below in Table 3.

TABLE 3
Subject Samples Results Megestrol Acetate in Plasma (ng/mL)

| time | Sub 1 | Sub 2 | Sub 3 | Sub 4 | Sub 5 | Sub 6 | Sub 7 | Sub 8 | Sub 9 | Sub 10 |
|---|---|---|---|---|---|---|---|---|---|---|
| 0 | 0.492 | 6.48 | 1.98 | 4.44 | 0.426 | 8.58 | 0.851 | <0.100 | 1.40 | n. report. |
| 1 | 7.82 | 32.9 | 9.32 | 37.4 | 4.98 | 44.0 | 7.82 | 19.5 | 14.4 | 23.5 |
| 2 | 2.74 | 28.1 | 5.24 | 18.7 | 2.75 | 32.1 | 4.83 | 10.6 | 9.21 | 17.2 |
| 4 | 1.01 | 9.89 | 3.04 | 7.22 | 0.959 | 12.6 | 1.99 | 3.68 | 4.02 | 6.93 |
| 8 | 0.607 | 6.98 | 2.66 | 4.70 | 0.521 | 7.61 | 1.47 | 1.41 | 1.64 | 4.95 |
| 12 | 0.413 | 5.84 | 1.98 | 3.05 | 0.353 | 5.80 | 1.59 | 0.908 | 1.59 | 2.85 |
| 24 | 0.399 | 4.40 | 1.80 | 2.17 | 0.344 | 4.04 | 0.771 | 0.459 | 1.09 | 1.77 |
| 36 | 0.504 | 5.04 | 1.99 | 3.58 | 0.409 | 6.58 | 0.592 | 1.34 | 1.30 | 2.60 |
| 48 | 0.463 | 3.58 | 1.48 | 2.90 | 0.267 | 5.65 | 0.564 | 0.796 | 1.31 | 1.63 |

The time measured represents the number of hours passed post dosing. Subjects 2, 4, 6, 8 and 10 all received a 6 mg dose of megestrol acetate. Subjects 1, 3, 5, 7 and 9 all received a 1.5 mg dose of megestrol acetate. For the predose sample of subject 10, no value can be reported (the deviation of two measurements did not fulfill the acceptance criterion for reanalyzed samples). A third measurement was not possible due to insufficient sample. During the study, 3 series of plasma calibration curves were measured (prevalidation run not considered). The statistics on regression parameters and backcalculated calibration standards are presented in the following tables.

TABLE 4
Statistics on Megestrol Acetate Regression Parameters

| | Intercept | Slope | r² |
|---|---|---|---|
| n | 3 | 3 | 3 |
| mean | 0.00255 | 0.15510 | 0.99678 |
| s | 0.00514 | 0.02465 | 0.00252 |
| C.V. [%] | | 15.89 | |

TABLE 5
Statistics on Backcalculated Megestrol
Acetate Calibration Standards

| | Cal. std. nominal conc. [nglmL] | | | | | | | |
|---|---|---|---|---|---|---|---|---|
| | 0.100 | 0.200 | 0.500 | 1.00 | 2.00 | 5.00 | 10.0 | 20.0 |
| Number | 3 | 3 | 3 | 3 | 3 | 3 | 3 | 3 |
| Mean (calc.) | 0.100 | 0.201 | 0.481 | 1.01 | 2.04 | 5.13 | 9.73 | 20.0 |
| s | 0.00427 | 0.0107 | 0.0422 | 0.0198 | 0.0843 | 0.287 | 0.377 | 0.860 |
| C.V. [%] | 4.26 | 5.32 | 8.77 | 1.96 | 4.12 | 5.59 | 3.87 | 4.30 |
| Bias [%] | 0.29 | 0.48 | −3.71 | 0.76 | 2.22 | 2.62 | −2.70 | 0.04 |

Quality Control Samples

The inter-assay accuracy and precision data were calculated from 6 sets of QC samples. The accuracy (expressed as bias) and the precision (expressed as coefficient of variation CV.) data are shown in table 6.

TABLE 6
Inter-Assay Accuracy and Precision for
Megestrol Acetate QC Samples

| | Nominal QC | | |
|---|---|---|---|
| | conc. [ng/mL] | | |
| | 0.25 | 1.5 | 15 |
| Number | 6 | 6 | 6 |
| Mean (calc.) | 0.254 | 1.41 | 15.4 |
| s | 0.0337 | 0.120 | 1.29 |
| C.V. [%] | 13.28 | 8.47 | 8.41 |
| Bias [%] | 1.40 | −5.70 | 2.51 |

The QC sample results were acceptable. As it was necessary to dilute some samples, diluted QC-samples with dilution factor 2 and dilution factor 4 have also been measured.

In the specification, there has been disclosed typical preferred embodiments of the invention and, although specific terms are employed, they are used in a generic and descriptive sense only and not for purposes of limitation of the scope of the invention being set forth in the following claims.

What is claimed is:

1. A method of treating endometrial hyperplasia comprising:
administering a therapeutic amount of an estrogenic compound to a subject; and
administering a therapeutic amount of a progestational agent of less than 20 mg.

2. The method according to claim 1, wherein said progestational agent is selected from the group consisting of megestrol acetate, laevo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynoestrenol, quingestanol acetate, medrogestone, norgestrienone, dimethisterone, ethisterone, and cyproterone acetate.

3. The method according to claim 1, further comprising administering an androgen compound in a daily dose.

4. The method according to claim 2, wherein said megestrol acetate is continuously and uninterruptedly administered to said subject.

5. The method according to claim 2, wherein said megestrol acetate is administered in doses ranging from 1 mg to less than 20 mg.

6. The method according to claim 2, wherein said megestrol acetate is administered in doses ranging from 1.5 mg to 16 mg.

7. The method according to claim 2, wherein said megestrol acetate is administered in a 6 mg dose.

8. The method according to claim 1, wherein the estrogenic compound comprises a mixture of estrogenic compounds, wherein said mixture comprises salts of conjugated estrone, conjugated equilin, conjugated Δ^{8,9}-dehydroestrone, conjugated 17α-estradiol, conjugated 17α-dihydroequilin, conjugated 17β-dihydroequilin, conjugated 17β-estradiol, conjugated equilenin, conjugated 17α-dihydroequilenin, and conjugated 17β-dihydroequilenin.

9. The method according to claim 1, the estrogenic compound comprises a mixture of estrogenic compounds wherein said conjugated estrogens is selected from the group consisting of the sodium sulfate esters of estrone, equilin, 17α-dihydroequilin, 17β-dihydroequilin and 17α-estradiol.

10. A method of treating vasomotor symptoms comprising:
administering a therapeutic amount of an estrogenic compound to a subject; and
administering a therapeutic amount of a progestational agent of less than 20 mg.

11. The method according to claim 10, wherein said progestational agent is selected from the group consisting of megestrol acetate, laevo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynoestrenol, quingestanol acetate, medrogestone, norgestrienone, dimethisterone, ethisterone, and cyproterone acetate.

12. The method according to claim 10, further comprising administering an androgen compound in a daily dose.

13. The method according to claim 11, wherein said megestrol acetate is continuously and uninterruptedly administered to said subject.

14. The method according to claim 11, wherein said megestrol acetate is administered in doses ranging from 1 mg to less than 20 mg.

15. The method according to claim 10, wherein the estrogenic compound comprises a mixture of estrogenic compounds, wherein said mixture comprises salts of conjugated estrone, conjugated equilin, conjugated Δ^{8,9}-dehydroestrone, conjugated 17α-estradiol, conjugated 17α-dihydroequilin, conjugated 17β-dihydroequilin, conjugated 17β-estradiol, conjugated equilenin, conjugated 17α-dihydroequilenin, and conjugated 17β-dihydroequilenin.

16. The method according to claim 10, the estrogenic compound comprises a mixture of estrogenic compounds wherein said conjugated estrogens is selected from the group consisting of the sodium sulfate esters of estrone, equilin, 17α-dihydroequilin, 17β-dihydroequilin and 17α-estradiol.

17. A method of treating vasomotor symptoms comprising:
administering a first dose of a therapeutic amount of an estrogenic compound to a subject;
administering a second dose of a therapeutic amount of an estrogenic compound at a later time period to the subject, said second dose comprising a lower dosage of said therapeutic amount of an estrogenic compound than said first dose; and
administering a therapeutic amount of a progestational agent of less than 20 mg.

18. The method according to claim 17, wherein said progestational agent is selected from the group consisting of megestrol acetate, laevo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynoestrenol, quingestanol acetate, medrogestone, norgestrienone, dimethisterone, ethisterone, and cyproterone acetate.

19. The method according to claim 17, wherein said second dose of an estrogenic compound is administered between 2 weeks and 12 weeks after the first dose of an estrogenic compound.

20. The method according to claim 17, wherein said second dose of an estrogenic compound is administered between 4 weeks and 8 weeks after the first dose of an estrogenic compound.

21. The method according to claim 17, wherein said vasomotor symptoms are selected from the group of hot flashes, cold flashes, night sweats, day sweats, dry vagina, dry hair and skin, insomnia, bladder problems and moodiness.

22. The method according claim 17, wherein said first dose is continuously and uninterruptedly administered to said subject for a predetermined period of time and then said second dose is continuously and uninterruptedly administered to said subject.

23. The method according to claim 17 further comprising:
administering a third dose of a therapeutic amount of an estrogenic compound at a later time period to the subject than that of said second dose, said third dose comprising a lower dosage of said therapeutic amount of an estrogenic compound than said second dose.

24. The method according to claim 18, wherein said megestrol acetate is administered in doses ranging from 1 mg to less than 20 mg.

25. The method according to claim 17, wherein the estrogenic compound comprises a mixture of estrogenic compounds, wherein said mixture comprises salts of conjugated estrone, conjugated equilin, conjugated Δ^{8,9}-dehydroestrone, conjugated 17α-estradiol, conjugated 17α-dihydroequilin, conjugated 17β-dihydroequilin, conjugated 17β-estradiol, conjugated equilenin, conjugated 17α-dihydroequilenin, and conjugated 17β-dihydroequilenin.

26. The method according to claim 17, the estrogenic compound comprises a mixture of estrogenic compounds wherein said conjugated estrogens is selected from the group consisting of the sodium sulfate esters of estrone, equilin, 17α-dihydroequilin, 17β-dihydroequilin and 17α-estradiol.

27. A method of treating menopause comprising:
administering a therapeutic amount of an estrogenic compound to a subject; and
administering a therapeutic amount of a progestational agent of less than 20 mg.

28. The method according to claim 27, wherein said progestational agent is selected from the group consisting of megestrol acetate, laevo-norgestrel, dl-norgestrel, norethindrone (norethisterone), norethindrone (norethisterone) acetate, ethynodiol diacetate, dydrogesterone, medroxyprogesterone acetate, norethynodrel, allylestrenol, lynoestrenol, quingestanol acetate, medrogestone, norgestrienone, dimethisterone, ethisterone, and cyproterone acetate.

29. A method for treating a patient afflicted with vasomotor symptoms, comprising administering an estrogenic compound to said patient for at least two cycles of a cyclical dosing schedule, wherein the first cycle comprises a dosing period of 4 to 12 weeks, in which the estrogenic compound is administered daily, at a dose of 0.625 to 1.5 mg/day, followed by a second cycle comprising a dosing period that can last for an indeterminate period of time in which an estrogenic compound is administered daily, at a dose of 0.05 to 0.625 mg/day and by administering megestrol acetate daily at a dose of 6 mg/day.

30. A method of treating endometrial hyperplasia comprising:
administering a dose of a therapeutic amount of an estrogenic compound to a subject;
administering a dose of a therapeutic amount of less than 20 mg of megestrol acetate to a subject; and
administering a second dose of a therapeutic amount of megestrol acetate at a later time period to the subject, said second dose comprising a lower dosage of said therapeutic amount of megestrol acetate than said first dose.

31. The method according to claim 30, wherein said megestrol acetate is administered in doses ranging from 1 mg to less than 20 mg.
